SEQUENCE LISTING (6 sequences)

SEQ ID NO: 1 - protein, 862 aa, Nicotiana tabacum
  ref <308>: DATABASE ACCESSION NUMBER: X84040
  ref <309>: DATABASE ENTRY DATE: 1995-07-16
MFLEKIVDAITGKDDGKKVKGTVVLMKKNVLDFTDINASVLDGVLEFLGRRVSLELISSVNADPANGLQGKRSKAAYLENWLTNSTPIAAGESAFRVTFDWDDEEFGVPGAFIIKNLHFSEFFLKSLTLEDVPNHGKVHFVCNSWVYPANKYKSDRIFFANQAYLPSETPDTLRKYRENELVTLRGDGTGKLEEWDRVYDYAYYNDLGDPDKGQDLSRPVLGGSSEYPYPRRGRTGRKPTKTDPNSESRIPLLMSLDIYVPRDERFGHIKLSDFLTFALKSIVQLLLPEFKALFDSTHNEFDSFEDVLKLYEGGIKLPQGPLLKAITDSIPLEILKELLRSDGEGLFKYPTPQVIQEDKTAWRTDEEFGREMLAGVNPVIISRLQEFPPKSKLDPKIYGNQNSTITREQIEDKLDGLTIDEAIKTNRLFILNHHDILMPYLRRINTSTDTKTYASRTLLFLQDNGTLKPSAIELSLPHPDGDQFGAVSKVYTPADQGVEGSIWQLAKAYAAVNDSGVHQLISHWLNTHAAIEPFVIATNRQLSALHPIYKLLHPHFRETMNINALARQILINGGGLLELTVFPAKYSMEMSAVVYKDWVFPEQALPTDLIKRGVAVEDSSSPLGIRLLIQDYPYAVDGLKIWSAIKSWVTEYCNYYYKSDDAVQKDTELQAWWKELREEGHGDKKDEPWWPKMQTVQELIDSCTITIWIASALHAAVNFGQYPYAGYLPNRPTLSRNFMPEPGSPEYEELKTNPDKVFLKTITPQLQTLLGISLIEILSRHSSDTLYLGQRESPEWTKDQEPLSAFARFGKKLSDIEDQIMQMNVDEKWKNRSGPVKVPYTLLFPTSEGGLTGKGIPNSVSI

SEQ ID NO: 2 - DNA, 3390 bp, Artificial sequence
  construct CaMV 35S-LOX
  promoter (1)..(532): CaMV 35S promoter
  CDS (543)..(3131): Tobacco LOX1 coding sequence
  terminator (3138)..(3390): Nos terminator
ccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagaccttccaaccacgtcttcaaagcaagtggattgatgtgatatctccactgacgtaagggatgacgcacaatcccactatccttcgcaagacccttcctcctataaggaagttcatttcatttggagaggacacgcggtacccaaaatgtttctggagaagattgtggatgcaatcacagggaaagatgatggaaaaaaggtaaaaggaacagtggttttgatgaagaaaaatgttttggattttactgatattaatgcctcagttcttgatggagttcttgagttccttggtcggagggtctctctcgagttgatcagttctgttaatgctgatcctgcaaatggtttacaagggaaacgcagcaaagcagcatatttggagaactggctaacaaatagcaccccaatagcagcaggtgaatcagcatttagagtcacatttgattgggatgatgaggaatttggagttccaggagcattcattatcaagaacttgcattttagtgagttcttcctcaagtcactcacccttgaagatgttcctaatcatggcaaagttcattttgtctgtaattcttgggtttatcctgctaataaatataagtcagatcgcatcttcttcgcgaatcaggcttatctaccaagtgaaacaccagacacattgcgaaaatacagagaaaatgaattagtaaccttaagaggagatggaactggaaagcttgaggaatgggatagagtttatgactatgcttactacaatgacttgggtgatccagacaaaggccaagatttgtcaaggcctgtcttaggaggatcttctgagtacccgtatcctcgtagaggcaggacaggccgcaaaccaaccaaaacagatcctaattccgagagcaggattccattgcttatgagcttagacatatatgtgccaagggacgagcgatttggtcatataaagttgtcagacttcttgacatttgctttgaaatccattgtgcagttgcttctccctgagtttaaggctttgttcgatagcacgcataatgagtttgatagttttgaggatgtacttaaactgtatgaaggaggaatcaagttgcctcaaggccctttgttgaaagccattactgatagcattcctttagagatactaaaagaactccttcgaagtgatggtgaaggcctatttaagtacccaactcctcaggttattcaagaggataaaactgcatggaggacggatgaagaatttgggagagaaatgttggcgggagtcaatcctgtcataatcagtagactccaagaattccctccgaaaagcaagttggatcctaaaatatatggcaaccaaaacagtacaattaccagagagcagatagaggataagttggatggactaacaattgatgaggcaatcaagactaacagactattcatattgaaccatcatgatatccttatgccatacttgaggagaattaacacgtcgacagacacaaaaacctatgcctcaagaactctgctcttcttgcaagataatggaactttgaagccatcagcaattgaactaagcttgccacatccagacggagatcaatttggcgctgttagcaaagtatatacaccagctgatcaaggtgttgaaggttctatctggcagttggccaaagcctatgcagcagtgaatgattcgggcgttcatcaactcatcagtcactggttgaatacacatgcagcgatagagccattcgtgatcgcaacaaataggcaactaagcgcgcttcaccctatttataagcttctccaccctcatttccgtgagacgatgaacataaatgctttagcaagacagatcttgatcaacggtggtggacttcttgagttgacagtttttccggccaaatattccatggaaatgtcagcagtagtttacaaagactgggttttccctgaacaagcacttcctactgatctcatcaaaagaggagtagctgttgaggactcgagctccccacttggcattcgattactgattcaggactatccatatgctgttgatgggttgaaaatttggtcagcaattaaaagttgggtaactgaatactgcaactactattacaaatcagatgatgcggttcaaaaagacactgaactccaagcctggtggaaggaactccgcgaagagggacacggtgacaagaaagatgagccttggtggcctaaaatgcagacagtgcaagaattgatagactcttgcaccatcacaatatggatagcttcagcacttcatgcagcagtcaatttcgggcaatacccttatgctggttatctccctaatcgccctacattaagccgaaatttcatgccagagccaggaagtcctgagtatgaagagctcaagacaaatccggataaggtattcctcaaaacaatcactcctcagctgcagacactgcttggcatttccctcatagagatcttgtcaaggcattcttcggatacactttacctcgggcaaagggaatcacctgaatggacaaaggatcaagaaccactttcagcttttgcgaggtttggaaagaagctgagtgatatcgaggatcagattatgcagatgaatgtcgatgagaaatggaagaacaggtcgggtcctgttaaagttccatacaccttgctcttccccacaagtgaaggaggacttactggcaaaggaattcctaacagtgtgtcaatatagaactttcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacg

SEQ ID NO: 3 - DNA, 30 bp, Artificial sequence
  Artificial sequence description: Sense primer
gttatcaaacagtttaaaatgtttctggag

SEQ ID NO: 4 - DNA, 22 bp, Artificial sequence
  Artificial sequence description: Reverse primer
tgatttaaagttctatattgac

SEQ ID NO: 5 - DNA, 20 bp, Artificial sequence
  Artificial sequence description: Primer F
ggccatggagtcaaagattc

SEQ ID NO: 6 - DNA, 19 bp, Artificial sequence
  Artificial sequence description: Primer R
gctctggcatgaaatttcg

(19) United States
(12) Patent Application Publication
(10) Pub. No.: US 2004/0205842 A1
(43) Pub. Date: Oct. 14, 2004

(54) Lipoxygenase overexpression in plants and reduction in plant sensitivity to diseases and to attacks from pathogenic organisms

(76) Inventors: Laurent Mene-Saffrane, Ogeu-Les-Bains (FR); Marie-Therese Esquerre-Tugaye, Castanet-Tolosan (FR); Joelle Fournier, Muret (FR); Roland Beffa, St. Genis Laval (FR); Marie-Claire Grosjean-Cournoyer, Curis Au Mont D'Or (FR)

Correspondence Address: BAKER & BOTTS, 30 ROCKEFELLER PLAZA, NEW YORK, NY, 10112

(21) Appl. No.: 10/731,642

(22) Filed: Dec. 8, 2003

Related U.S. Application Data
(63) Continuation of application No. PCT/FR02/01943, filed on Jun. 6, 2002.

(30) Foreign Application Priority Data
Jun. 7, 2001 (FR) 01/07470
Nov. 7, 2001 (FR) 01/14358

(51) Int. Cl. A01H 1/00; C12N 15/82; C12N 9/02

(52) U.S. Cl. 800/279; 435/189; 435/468

(57) ABSTRACT

The present invention relates to methods for reducing plant sensitivity to diseases and attacks from pathogenic organisms. The methods according to the invention comprises overexpressing an inducible lipoxygenase in plants in order to improve their response to diseases and pathogens. The invention also relates to expression cassettes for overexpressing lipoxygenases in plants, and also to transformed plants.

This application is a continuation of International Patent application No. PCT/FR02/01943 filed Jun. 6, 2002 and published as WO 02/099112 on Dec. 12, 2002, which claims priority to French Patent Application No. FR 01/07470 filed Jun. 7, 2001 and French Patent Application No. FR 01/14358 filed Nov. 7, 2001.

### BACKGROUND OF THE INVENTION

The present invention relates to methods for reducing plant sensitivity to diseases and attacks from pathogenic organisms. The methods according to the invention comprise overexpressing a lipoxygenase in plants so as to reduce their sensitivity to diseases and attacks. The invention also relates to expression cassettes, to vectors and to transformed plants used in the methods according to the invention.

Lipoxygenases (LOXs) are ubiquitous enzymes in higher plants and mammals. They catalyze the dioxygenation of polyunsaturated fatty acids containing a (Z,Z)-1,4-pentadiene unit. In plants, substrates for the enzyme are linoleic acid (C18:2) and linolenic acid (C18:3), which are two major constituents of cell membranes. These polyunsaturated fatty acids are generally complexed in the form of membrane-bound phosphoglycerolipids and are only accessible to LOXs after the action of a phospholipase type A₂ or lipolytic acyl hydrolases. Recent studies suggest, however, that certain LOXs may, under certain circumstances, oxidize esterified fatty acids and membrane-bound lipids (1-4). Arachidonic acid, which is not detectable in plants, but which is part of the membrane-bound constituents in oomycetes, is also a substrate for plant LOXs (5).

LOXs are classified according to the position of the carbon onto which the molecular oxygen is preferentially inserted. In plants, 13-LOXs and 9-LOXs are distinguished; the same enzyme can, however, use one or other position, indifferently or else with a preference for a position. This specificity can be modified according to the conditions of pH and of O₂ concentration of the medium (4). The position specificity of a LOX cannot be directly predicted according to its primary sequence, even though some structural elements related to this property are now known (6, 7).

The products formed in the reaction are fatty acid hydroperoxides, which are very reactive and capable of causing, via free-radical reactions, degradation of the major constituents of the cell (lipids, proteins, nucleic acids) (8). Fatty acid hydroperoxides are rapidly converted into a series of compounds having diverse biological activities. All the products derived from polyunsaturated fatty acids via oxygenation are collectively referred to as oxylipins.

Plant LOXs have been associated with varied physiological processes, on the basis of profiles of gene expression and of enzyme activity. Thus, it has been proposed that LOXs are involved in regulating seed maturation, germination, fruit maturation, and leaf and flower senescence. The precise contribution of LOXs to these processes remains to be determined however. An important role is also attributed to LOXs in response to stress, in particular injury, and parasitic attacks (4, 5, 9). Strong induction of LOX gene expression is thus measured in many monocotyledon or dicotyledon plants when they interact with bacteria, viruses or fuingi, and also after mechanical injury or injury caused by insects on the leaves.

This diversity in the biological functions may be provided due to the presence of various isoenzymes, exhibiting highly varied mechanisms of regulation and tissue and subcellular locations, according to the species and isoforms under consideration (5). Added to this is the diversity of the oxylipins generated from the products of the LOX, which is modulated according to the type of hydroperoxide formed and to the nature of the enzymes which metabolize them.

The fatty acid hydroperoxides generated by the LOX are in fact converted according to several distinct enzyme pathways (4). The hydroperoxide lyase (HPL) pathway catalyzes cleavage of 13- or 9-hydroperoxides of fatty acids so as to achieve the synthesis of volatile C6- or C9-aldehydes and of C12 or C9 short-chain acids, such as 12-oxo-trans-9-dodecenoic acid, the precursor of traumatic acid. C6-aldehydes play an important role in plant fragrance, but some, such as trans-2-hexenal, also have antimicrobial properties (10). It has recently been shown that trans-2-hexenal can act as a signal molecule for defense gene activation (11). Traumatic acid, also referred to as a wound hormone, is thought to have a role in tissue healing by promoting cell division at sites of injury (5).

A second enzyme pathway involved in the metabolism of fatty acid hydroperoxides produced by LOX concerns allene oxide synthase (AOS). This enzyme catalyzes the dehydration of 13-hydroperoxilinolenic acid and forms an allene oxide, which is the precursor of the jasmonic acid. Jasmonic acid is a key molecule of plant signaling mechanisms for activating many defense genes (12), among which are the genes encoding protease inhibitors active against insects, and also many PR proteins (chitinase, defensin, thionin, glucanase). The role of jasmonates as a signal molecule has recently been underlined by various experiments showing that Arabidopsis mutants or tomato mutants affected in terms of the biosynthesis of these molecules or the distinction thereof, exhibit a deficiency in defense gene induction (13), greater sensitivity to organisms which are normally nonpathogenic for wild-type plants (14) or reduced resistance to insects (15).

A third enzyme pathway for conversion of hydroperoxides concerns the formation of divinyl ether fatty acids. These compounds, which can be formed from 13- or 9-hydroperoxides, have been isolated from various plants. The first divinyl ether synthase (DES) was recently cloned in the tomato (16). The divinyl ether fatty acids produced from 9-hydroperoxides, colneleic acid and colnelenic acid, exhibit antifungal properties, in particular with respect to Phytophthora infestans (17).

Other modifications of the products of the reaction catalyzed by LOX concern epoxidations with epoxygenases and peroxygenases. This metabolic pathway allows the synthesis of molecules with antimicrobial activity and may be involved in the synthesis of cut in monomers, which, besides their structural function, can also induce the activity of defense genes (18).

The fatty acid hydroperoxides produced by LOX can, finally, generate the formation of free radicals involved in membrane degradation mechanisms associated with cell death (5).

All these data therefore show that the initial activity of LOX is essential for the synthesis of a set of molecules, some of which exhibit antimicrobial activities or are involved in the signaling leading to defense.

LOX activity increases notably in tobacco in response to elicitors (19, 20). The isolation of a LOX complementary DNA clone, in particular pTL-J2 (21), has made it possible to characterize the corresponding tobacco gene, called LOX1. DNA/RNA hybridization experiments (Northern hybridization) have shown that LOX1 is expressed in tobacco cells in culture subsequent to the application of elicitor and in tobacco plants inoculated with the oomycete Phytophthora parasitica nicotianae, Ppn (22). The transcripts corresponding to this gene are not detectable in normal plants or untreated cells. A biochemical study shows that, in vitro, the LOX of elicited tobacco cells allows the production of 9- and 13-hydroperoxides of fatty acid, with preferential insertion of the molecular oxygen in the 9-position (20).

Subsequent to root inoculation of tobacco with Ppn, strong induction of expression of the gene is measured. In the case of an incompatible interaction (resistant plant/avirulent race of microorganism), early induction of the activity of the gene is measured, with a maximum accumulation of transcripts at 24 hours (22). Induction of the gene is later and smaller in amplitude in the case of a compatible interaction (sensitive plant/virulent race), as has also been observed in other gene-for-gene models, such as interactions between the tomato and Pseudomonas syningae (23), rice and Magnaporthe grisea (24), and the potato and Phytophthora infestans (25). Expression of the LOX1 gene is not detected in the tissues of normal tobacco plants, with the exception of flowering plants for which the LOX transcripts are detected in low amount in the petals and sepals, and young germinations (22, 26). In the latter case, transient expression of the LOX1 gene is detected between the second and fourth days after the beginning of germination.

Villalba et al. (27) have shown that an elicitor glycoprotein isolated from Ppn walls, called CBEL for Cellulose Binding Elicitor Lectin, induces the LOX1 gene when it is infiltrated into the foliar mesophyll of tobacco plants. The LOX1 transcripts appear during the first four hours following infiltration and reach a maximum level at 12 hours. A polysaccharide derived from marine algae, λ-carraghenan, also induces the LOX1 gene when it is applied to tobacco plants by infiltration into the foliar mesophyll (28).

In tobacco cells, expression of the LOX1 gene is detected subsequent to eliciting treatments. In the case of Ppn elicitor (wall extract), induction of expression of the LOX1 gene is detected within the first two hours after treatment, with a maximum accumulation at 24 hours (22). Cryptogein, an elicitor peptide of Phytophthora cryptogea, an oomycete for which tobacco is not a host, and Colletotrichum lindemuthianum endopolygalacturonase, also make it possible to induce the LOX1 gene (26). It has been shown, moreover, that induction of the LOX1 gene occurs earlier than that of defense genes such as those encoding PR proteins, namely chitinases and β1-3-glucanases, suggesting that the LOX pathway has a potential role in the signal transduction triggering the defense reactions (26). LOX1 expression in cells is also inducible with methyl jasmonate (22, 29), thus indicating possible self-amplification of this pathway, whereas no accumulation of LOX transcripts is detected after the application of salicylic acid (22).

Analysis of expression of the LOX1 gene shows that the induction of LOX constitutes an early response of the plant to infection with Ppn, suggesting a role in the establishment of resistance. This potential role has been confirmed through obtaining transgenic plants expressing the complementary DNA of the gene in an antisense orientation. In fact, these plants, originating from the 46-8 line normally resistant to Ppn race 0, exhibit greatly reduced levels of LOX activity and have lost their ability to trigger the incompatible reaction (30, 31). This experiment clearly shows that expression of the LOX gene is necessary to establish the resistant state in the gene-for-gene interaction between tobacco and Ppn. The LOX antisense plants are also more sensitive to another pathogenic fungus of tobacco, Rhizoctonia solani (30, 31). In parallel, Rustérucci et al. (32) have shown that 9-hydroperoxides accumulate during the hypersensitive-type response of tobacco to cryptogein, and that this accumulation is necessary for the development of this reaction. In another Solanacea, the potato, oxylipins derived from the 9-LOX pathway, in particular colneleic acid and colnelenic acid, preferentially accumulate in cells in culture in response to a P. infestans elicitor (33). All these data suggest a major role for the 9-LOX pathway in Solanaceae, including tobacco, in the response to pathogenic agents, in particular oomycetes. In these plants, the 13-LOX pathway contributes to the response to pathogenic agents, as shown by the early synthesis of jasmonates (29); it is also involved in the response to injury and to insects. Thus, potato plants expressing a 13-LOX antisense construct have been found to be more sensitive to insect attack (34).

Few attempts at overexpressing a LOX in plants have been reported in the literature. In fact, the expression of a LOX poses questions of feasibility due to the potentially toxic properties of LOXs, if they directly oxidize biomembranes, and of the products which they form.

In tobacco, introduction of soybean LOX2 under the control of a chimeric promoter, formed by fusion of the cauliflower mosaic virus (CaMV) 35S promoter and an enhancer isolated from the alfalfa mosaic virus, has made it possible to produce transgenic calluses exhibiting a LOX activity which is approximately 2 times greater than that of calluses transformed with the vector lacking LOX sequence. These calluses are capable of forming 3 to 6 times as many volatile aldehydes, products of the 13-LOX pathway via a 13-HPL, as the control calluses. Transgenic plants were regenerated from these calluses but, although these plants exhibited a high accumulation of the heterologous protein, their LOX activity is no different from that of control plants. However, they form larger amounts of volatile aldehydes than control plants (35). More recently, a 13-LOX specific for the lipid bodies of cucumber seeds has been expressed in tobacco (36). The heterologous protein accumulates throughout the plant, in transgenic tobacco plants, and in particular in the seeds, where it is essentially located in the lipid bodies as in the cucumber. Its presence results in a qualitative modification of the LOX activity of the transgenic plants, both in vitro and in vivo. An Arabidopsis chloroplast 13-LOX, AtLOX2, has been used in a construct in the sense orientation, under the control of the CaMV 35S promoter, to transform garden arabis plants. The authors focus, however, on the transformation events which led to a decrease in LOX activity by cosuppression (37). Finally, in the lentil, transient transformation of protoplasts with a construct containing a lentil LOX under the control of the CaMV 35S promoter produced a 20% increase in the LOX activity of these protoplasts (38). None of the transformed plants or cultures described above was tested with respect to a response relating to attack from pathogenic agents. Moreover, the experiments carried out essentially concern 13-LOXs.

In addition, the hypotheses concerning the participation of lipoxygenases in plant defense mechanisms are essentially based on the biological activities of certain oxylipins (39). Now, the biosynthesis of most oxylipins, and in particular jasmonic acid and the volatile aldehydes derived from the 13-LOX pathway, and the divinyl ethers derived from the 9-LOX pathway, requires the activity of enzymes which metabolize the LOX products, such as AOS, HPL or DES, beyond the LOX itself. Expression of the genes corresponding to these enzymes is often inducible. In tomato and Arabidopsis thaliana, the expression of genes encoding AOS and HPL is wound-inducible (40-42). Consequently, the action of only lipoxygenases does not therefore appear to be sufficient to trigger plant resistance mechanisms.

Cultivated plants are subjected to attack by many pathogenic organisms, such as viruses, bacteria and fungi, but also pests such as insects. These attacks weaken the plants and decrease their crop yields. There is therefore a considerable need to increase the resistance mechanisms of plants and to decrease their sensitivity to diseases and attacks from parasitic or pathogenic organisms. The mechanisms of responsive plants to attacks from pathogenic agents have been the subject of many studies. It is now commonly accepted that the lipoxygenase pathway in plants contributes to their system of defense and to establishing a state of resistance through the oxylipin pathway in particular.

However, knowledge of these metabolic pathways and of the lipoxygenases has not made it possible to develop methods for directly increasing plant resistance. This problem is addressed by the present invention since it has now been noted that overexpression of a lipoxygenase in plants directly reduces plant sensitivity to diseases and attacks from pathogenic agents.

### SUMMARY OF THE INVENTION

The present invention provides sequences, proteins, nucleic acids, expression cassettes, vectors, and methods for increasing lipoxygenase activity in plant cells. In addition, the invention provides transgenic plant cells that have higher levels of lipoxygenase activity than corresponding plant cells lacking the transgene. The invention also provides transgenic plants that have higher levels of lipoxygenase activity than corresponding plants lacking the transgene. The invention further provides transgenic plant cells that are more resistant to at least one pathogen or herbivore than corresponding plant cells lacking the transgene, wherein the transgene comprises a lipoxygenase gene. The invention also provides transgenic plants comprising that are more resistant to at least one pathogen or herbivore than corresponding plants lacking the transgene, wherein the transgene comprises a lipoxygenase gene. The transgenic plant cells and plants of the invention are phenotypically the same or substantially the same as corresponding plant cells and plants lacking the transgene.

### BRIEF DESCRIPTION OF THE DRAWINGS

FIG. 1 FIG. 1 presents the construct 35S-9-LOX used to transform tobacco. The 9-LOX (LOX1) coding sequence was obtained by PCR amplification and then inserted between the cauliflower mosaic virus 35S promoter (35S) and the Agrobacterium tumefasciens nopaline synthase terminator (tnos). This vector also comprises the neomycin phosphotransferase (NPTII) gene which confers resistance to kanamycin in bacteria and plants. “F” (“sense 35S”) denotes the sense primer and “R” (“reverse LOX 1”) denotes the reverse primer.

FIG. 2 FIG. 2 is a histogram representing the measurement of LOX specific activity in the tobacco plant stems in nKAT/mg of protein. 46-8 WT and 49-10 WT denote the parental lines. S46-21, S46-26 and S49-18 denote the transgenic lines. In fact, prior to Ppn inoculation tests, the LOX specific activity and also the level of corresponding transcripts were analyzed for 12-week-old tobacco plant stems. It is observed that the level of LOX specific activity in the three transgenic lines selected is significantly higher than that measured in the corresponding parental lines.

FIG. 3 FIG. 3 is a histogram representing the measurement of lesion length in mm. These measurements were made 48 hours (48h) or 72 hours (72h) after inoculation. The numbers in brackets correspond to the independent repetitions carried out. The two lines S46-21 and S46-26, inoculated with Ppn 1, exhibit lesion length significantly shorter than those obtained with the same pathogenic agent in the wild-type parent, 46-8 WT. Similarly, the line S49-18 exhibits shorter lesions than those measured in its wild-type parent, 49-10 WT, when these two lines are inoculated with Ppn 0. These differences are significant at 48 hours and at 72 hours after inoculation.

### DESCRIPTION OF THE SEQUENCE LISTING

SEQ ID NO:1: Tobacco lipoxygenase (LOX1).

SEQ ID NO:2: CaMV 35S promoter—coding sequence of tobacco LOX1 gene—nos terminator expression cassette.

SEQ ID NOS:3-6: Primers for PCR.

### DETAILED DESCRIPTION OF THE INVENTION

Unexpectedly, and although lipoxygenase is part of complex metabolic pathways, overexpression of a lipoxygenase in plants is sufficient to improve plant response to attacks from pathogens. In addition, overexpression of the lipoxygenase does not substantially affect the phenotype of the transformed plants, besides the novel acquired properties.

The present invention therefore discloses overexpression of a lipoxygenase in plants to reduce plant sensitivity to diseases and attacks from pathogenic organisms or pests. A subject of the invention is also preferred expression cassettes for overexpressing the lipoxygenase in plants, and also plant cells and transformed plants.

The present invention relates to a method for reducing plant sensitivity to diseases and attacks from pathogenic organisms. This method comprises overexpressing a lipoxygenase in these plants.

The term “lipoxygenase” means an enzyme which catalyzes the dioxygenation of polyunsaturated fatty acids containing a (Z,Z)-1,4-pentadiene unit. In plants, the substrates for the enzyme are linoleic acid (C18:2) and linolenic acid (C18:3), which are two main constituents of cell membranes.

The term “overexpression” means that the lipoxygenase is expressed at a level greater than the expression level observed in a reference plant (not induced). This overexpression results in greater accumulation of the lipoxygenase gene transcripts and/or of the lipoxygenase itself, and in an increased lipoxygenase specific activity in the tissues of the plant. To reduce plant sensitivity to diseases and attacks from pathogenic organisms, it is important for the level of expression of the lipoxygenase to be greater than that of a reference plant when the attack on the pathogenic organism occurs.

Overexpression of the lipoxygenase makes it possible to reduce plant sensitivity to disease and attacks from pathogenic organisms. The expression “attack from pathogenic organisms” is in particular attacks on the plants from viruses, bacteria, fungi, oomycetes or insects.

In a preferred embodiment, the method comprises constitutively overexpressing a lipoxygenase in plants. The term “constitutively” denotes temporal and spatial expression of the lipoxygenase in plants in the methods according to the invention. The term “constitutively” means the expression of a lipoxygenase in the tissues of the plant throughout the life of the plant and in particular during its entire vegetative cycle. In a first embodiment, the lipoxygenase is expressed constitutively in all plant tissues. In a second embodiment, the lipoxygenase is expressed constitutively in the roots, the leaves, the stems, the flowers and/or the fruits. In another embodiment of the invention, the lipoxygenase is expressed constitutively in the roots, the leaves and/or the stems.

In a particular embodiment of the invention, the lipoxygenase is a lipoxygenase which is “inducible” in a reference plant. In the present invention, lipoxygenase which is “inducible” means a lipoxygenase which is not expressed, or is expressed at very low levels, and the expression of which is greatly induced in response to elicitors in the response to stress, to wounds, and in particular to diseases and attacks from pathogenic organisms.

In a preferred embodiment of the method according to the invention, the lipoxygenase preferably has 9-lipoxygenase activity. Lipoxygenases (LOX) are classified according to the position of the carbon onto which the molecular oxygen is preferentially inserted. In plants, 13-LOXs and 9-LOXs are distinguished. Methods for determining the specificity of lipoxygenase activity are described in the literature (Fournier et al., Plant J. 3:63-70, 1993; Homung et al., PNAS 96:4192-4197, 1999; Rustérucci et al., J. Biol. Biochem. 274:36446-36455, 1999).

Any lipoxygenase, the overexpression of which in plants makes it possible to reduce plant sensitivity to diseases and attacks by pathogen organisms, may be used in the methods according to the invention. Lipoxygenases are known to those skilled in the art, and other lipoxygenases may be identified using known techniques. By way of example, mention may in particular be made of potato (Kolomoiets M. V. et al., Plant Physiol. 124:1121-1130, 2000), tomato (Genbank AY008278) and potato tuber (Royo et al., J. Biol. Chem., 271:21012-21019, 1996; Casey, R., Plant Physiol., 107:265-266, 1995), lipoxygenases, almond seed lipoxygenase (Mita et al., Eur. J. Biochem., 268:1500-1507, 2001) and barley seed lipoxygenase (Van Mechelem et al., Biochem. Biophys. Acta, 1254:221-225, 1995).

The lipoxygenase is preferably a plant lipoxygenase.

In a particular embodiment of the invention, it is a Solanacea plant lipoxygenase. Among the Solanacea plants, mention may in particular be made of tobacco, tomato, potato or else pepper.

In another preferred embodiment of the invention, the lipoxygenase exhibits at least 80% homology with the tobacco lipoxygenase 1 (LOX1) of SEQ ID NO:1. Advantageously, the percentage homology will be at least 80%, 85%, 90%, 95%, and preferably at least 98%, and more preferably at least 99%, with respect to SEQ ID NO:1. The term “homolog” denotes a polypeptide which may exhibit a deletion, an addition or a substitution of at least one amino acid. The methods for measuring and identifying homologies between polypeptides or proteins are known to those skilled in the art. Use may, for example, be made of the UWGCG package and the BESTFITT program for calculating homologies (Devereux et al., Nucleic Acid Res. 12, 387-395, 1984). Preferably, the homologous lipoxygenases conserve the same biological activity as the tobacco lipoxygenase (LOX1) of SEQ ID NO:1. Preferably, these polypeptides therefore have a lipoxygenase activity, and even more preferably 9-lipoxygenase activity.

In an even more preferred embodiment, the methods according to the present invention use the lipoxygenase of SEQ ID NO:1.

Overexpression of the lipoxygenase in plants is carried out by transforming the plants with, or by applying to the plants, a molecule for stimulating lipoxygenase synthesis in the plant.

In a preferred embodiment of the invention, the lipoxygenase is overexpressed by integration into the genome of the plants of an expression cassette comprising a sequence encoding a lipoxygenase under the control of a promoter which is functional in plants.

According to the invention, the term “promoter” means the noncoding region of a gene involved in binding the RNA polymerase and with other factors which are responsible for initiating and regulating transcription leading to the production of an RNA transcript. The plant promoters which can be used in the methods according to the present invention are widely described in the literature.

The promoter is preferably a promoter which is constitutive in plants. The constitutive promoters which can be used in the method according to the invention are also well known to those skilled in the art.

As a promoter regulatory sequence in plants, use may be made of any promoter sequence for a gene which is naturally expressed in plants, such as, for example, “constitutive” promoters of bacterial, viral or plant origin. Mention may be made of bacterial promoters such as that of the octopine synthase gene or of the nopaline synthase gene, viral promoters, such as the cauliflower mosaic virus 35S promoter or the CSVMV promoter (WO 97/48819), and promoters of plant origin such as the histone gene promoter (EP0507698) or the promoter of a rice actin gene (U.S. Pat. No. 5,641,876). According to the invention, use may in particular be made, in combination with the promoter regulatory sequence, of other regulatory sequences, which are located between the promoter and the coding sequence, such as transcription activators (enhancers).

In a preferred embodiment of the invention, the constitutive promoter is the cauliflower mosaic virus 35S promoter.

In another embodiment of the invention, the constitutive expression or the overexpression of the lipoxygenase is obtained by transforming the plants so as to place a constitutive promoter or an enhancer sequence upstream of or in the vicinity of the lipoxygenase gene in the plants. Use may in particular be made of any regulative sequence for increasing the level of expression of lipoxygenase in plants.

Preferably, the lipoxygenase is overexpressed in the stems, the leaves and/or the roots of the plant.

According to the invention, the term “plant” means any differentiated multicellular organism capable of photosynthesis, in particular monocotyledons or dicotyledons, more particularly crop plants which may or may not be intended for animal or human food, such as maize, wheat, barley, sorghum, rapeseed, soybean, rice, cane sugar, beetroot, tobacco, cotton, etc.

Overexpression of the lipoxygenase can be obtained in any plant according to methods known to those skilled in the art.

In a particular embodiment of the invention, the plants are chosen from Solanaceae plants. Among the Solanacea plants mention will in particular be made of tobacco, tomato, potato or else pepper.

In a preferred embodiment of the invention, the lipoxygenase is overexpressed by integration into the plant genome of an expression cassette comprising a sequence encoding a lipoxygenase under the control of a promoter which is functional in plants. A polynucleotide encoding a lipoxygenase is inserted into an expression cassette using cloning techniques well known to those skilled in the art. This expression cassette comprises the elements required for transcription and translation of the sequences encoding the lipoxygenase in plants. Advantageously, this expression cassette comprises both elements for making the transformed plants produce the lipoxygenase and elements required for regulating this expression. The present invention also relates to preferred expression cassettes which can be used in the methods according to the invention.

In one embodiment, the present invention relates to expression cassettes which are functional in plant cells and plants, comprising a promoter having constitutive activity in plants, controlling the expression of a polynucleotide encoding a lipoxygenase at least 90% homologous to the lipoxygenase of SEQ ID NO:1. Advantageously, the percentage homology will be at least 80%, 85%, 90%, 95%, and preferably at least 98%, and more preferably at least 99%, with respect to SEQ ID NO:1. Preferably, this polynucleotide encodes a lipoxygenase having 9-lipoxygenase activity. More preferably, this polynucleotide encodes the lipoxygenase of SEQ ID NO:1. In a particular embodiment of the invention, the promoter is the cauliflower mosaic virus 35S promoter.

The expression cassettes according to the invention preferably comprise a terminator sequence. These sequences allow termination of transcription and polyadenylation of the MRNA. Any terminator sequences which is functional in plants can be used. For expression in plants, use may in particular be made of the Agrobacterium tumefaciens nos terminator, or else terminator sequences of plant origin, such as, for example, the histone terminator (EP 0 633 317), the CaMV 35S terminator or the tml terminator. The terminator sequences can be used in monocotyledon and dicotyledon plants.

The techniques for construction of these expression cassettes are widely described in the literature (see in particular Sambrook et al., Molecular Cloning: A Laboratory Manual, 1989).

Advantageously, the expression cassettes according to the present invention are inserted into a vector for replicating them or for transforming plants.

The present invention also relates to vectors for transforming plants, comprising at least one expression cassette according to the present invention. This vector may in particular consist of a plasmid or of a virus into which is inserted an expression cassette according to the invention. Many vectors have been developed for transforming plants with Agrobacterium tumefaciens. Other vectors are used for transformation techniques not based on the use of Agrobacterium. These vectors are well known to those skilled in the art and widely described in the literature. Preferably, the vectors of the invention also comprise at least one selection marker. Among the selection markers, mention may be made of genes for resistance to antibiotics, such as the nptII gene for kanamycin resistance (Bevan et al., Nature 304:184-187, 1983) and the hph gene for hygromycin resistance (Gritz et al., Gene 25:179-188, 1983). Mention will also be made of genes for tolerance to herbicides, such as the bar gene (White et al., NAR 18:1062, 1990) for tolerance to bialaphos, the EPSPS gene (U.S. Pat. No. 5,188,642) for tolerance to glyphosate or else the HPPD gene (WO 96/38567) for tolerance to isoxazoles.

The present invention also relates to vectors comprising an expression cassette according to the invention.

A subject of the invention is also a method for transforming plants with an expression cassette or a vector according to the invention.

According to the present invention, the plant transformation can be obtained by any suitable known means; plant transformation techniques are fully described in the specialist literature.

Certain techniques use Agrobacterium in particular for transforming dicotyledons. A series of methods consists in using a chimeric gene inserted into an Agrobacterium tumefaciens T1 plasmid or an Agrobacterium rhizogenes Ri plasmid as means of transfer into the plant. Other methods include bombarding the cells, protoplasts or tissues with particles to which the DNA sequences are attached. Other methods can also be used, such as microinjection or electroporation, or else direct precipitation with PEG.

Those skilled in the art will soon see appropriate methods as a function of the nature of the plant cell or of the plant.

The present invention relates to the transformed plant cells comprising an expression cassette and/or a vector according to the invention.

According to the invention, the term “plant cell” means any cell derived from a plant and which can constitute undifferentiated tissues such as calluses, differentiated tissues such as embryos, parts of plants, plants or seeds.

The present invention also relates to the transformed plants comprising an expression cassette, a vector and/or transformed cells according to the invention.

According to the invention, the term “plant” means any differentiated multicellular organism capable of photosynthesis, in particular monocotyledons or dicotyledons, more particularly crop plants which may or may not be intended for animal or human food, such as maize, wheat, barley, sorghum, rapeseed, soybean, rice, beetroot, tobacco, cotton, etc.

### EXAMPLES

### Example 1

### Biological Material

Wild-type tobacco plants (Nicotiana tabacum L.) of the two near-isogenic lines 46-8 (46-8 WT) and 49-10 (49-10 WT) were used (Helgeson et al., Phytopath. 62,1439-1443, 1972). These lines differ from one another by the presence, in the 46-8 WT line, of a locus for resistance to race 0 of Ppn. Thus, the 46-8 WT line is resistant to race 0 of Ppn and sensitive to race 1 of this pathogenic agent, whereas the 49-10 WT line is sensitive to the two races of Ppn.

The seeds of wild-type or transgenic lines are sterilized (Rancé et al., Plant Cell Report, 13, 647-651, 1994) and sown on solid MS medium supplemented, in the case of the transgenic lines, with kanamycin (50 μg.ml⁻¹). After 4 weeks of growth in vitro (40% hygrometry, constant temperature 25° C., light 60 μmol.m⁻².s⁻: 16h, dark: 8h), the kanamycin-resistant wild-type or transgenic plants are transferred onto vermiculite in a culture room (80% hygrometry, light 125 μmol.m⁻².s⁻¹: 16h, 25° C., dark: 8h, 18° C.).

The Ppn strains used correspond to the 1156 (race 0) and 1452 (race 1) isolates (Hendrix, J. W. & Apple, J. L., Tobacco Science 11, 148-150, 1967). The Ppn mycelium is cultured in the dark on a solid synthetic medium (Keen, N. T., Science 187, 74-75, 1975).

### Example 2

### Production of the CaMV 35S Promoter-LOX1 Coding Sequence-Nos Terminator Cassette (p35S-LOX1)

TL-J2 is a 2888 bp complementary DNA corresponding to the tobacco LOX1 gene induced by pathogenesis. The production of this complementary DNA is described by Véronési et al. (Véronési et al., Plant Physiol. 108, 1342, 1995), and its sequence is deposited in GenBank under the accession number X84040. This cDNA was used as a matrix for PCR amplification of the LOX1 coding sequence.

Primers were synthesized to amplify a 2.6 kb DNA fragment covering positions 49 to 2667 of the cDNA:

| | |
|---|---|
| Sense primer = | |
| 5′-GTTATCAAACAGTTTAAAATGTTTCTGGAG-3′ | (SEQ ID NO:3) |
| Reverse primer = | |
| 5′-TGATTTAAAGTTCTATATTGAC-3′ | (SEQ ID NO:4) |

These primers enable, in addition, the introduction of DraI sites (underlined in the primer sequence) upstream of the translation initiation: and downstream of the stop codon (indicated in bold characters in the primer sequence) of the LOX1 sequence.

The PCR reaction was carried out in a total volume of 25 μl, containing 50 ng of plasmid pTL-J2, 50 pmol of each of the sense and reverse primers above and 2.5 units of Pfu DNA polymerase (Stratagene Cloning Systems), and adjusted to 200 μM of each dNTP and 2 mM MgCl₂. After denaturation for 5 min at 94° C., the thermocycler program was made up of 20 cycles, each including denaturation for 1 min at 94° C., hybridization for 1 min at 50° C. and extension for 6 min at 72° C., followed by a final step of extension for 40 min at 72° C.

The DNA from this reaction was digested with DraI and separated on 0.8% agarose gel. The 2.6 kb blunt-ended fragment was purified from the gel (QiaEx II kit, Qiagen) and cloned into the Smal site of the vector pIPMO (Rancé et al., PNAS 6554-6559, 1998) between the CaMV 35S promoter and the 3′ untranslated region of the Agrobacterium tumefaciens nopaline synthase gene (nos terminator). This vector also comprises two copies of the neomycin phosphotransferase (NPTII) gene which confers kanamycin resistance in bacteria and plants. The ligation mixture was used to transform competent Escherichia coli XL1 Blue bacteria, and kanamycin-resistant colonies were selected and then screened for the presence of LOX sequence using the TL-J2 molecular probe. Positive colonies were cultured and the corresponding plasmids were purified. The orientation of the LOX1 sequence was examined for each of the plasmids by PCR using the following primers:

F primer, “sense 35S”: 5′-GGCCATGGAGTCAAAGATTC-3′ (SEQ ID NO:5) targeting nucleotide 6906-6925 of the CaMV 35S promoter (sequence available in Genbank under the accession number J02048).

R primer, “reverse LOX1”: 5′-GCTCTGGCATGAAATTTCG-3′ (SEQ ID NO:6) targeting nucleotides 2290-2272 (noncoding strand) of TL-J2.

The amplification reactions were carried out in the volume of 50 μl and comprising 100 ng of test plasmid, 10 pmol of each primer and 1 unit of Taq DNA polymerase in a medium adjusted to 200 μM of each dNTP and 1.5 mM MgCl₂. The thermocycler program included an initial denaturation step of 5 min at 94° C., and then 40 cycles each consisting of denaturation for 1 min at 94° C., hybridization for 1 min at 65° C. and extension for 2 min at 72° C., followed by a final elongation step for 10 min at 72° C. The reaction products were separated on 0.8% agarose gel. A plasmid for which the presence of an amplification product of the expected size (2.8 kb) indicated the sense orientation of the LOX sequence relative to the CaMV 35S promoter in the construct was selected. The LOX sequence and the junctions thereof with the promoter and the terminator were entirely sequenced. The plasmid thus verified is called p35S-LOX1. The sequence of the CaMV 35S-LOX1 construct is described in SEQ ID NO:2.

### Example 3

### Genetic Transformation of Tobacco

The plasmid p35S-LOX1 was mobilized in the Agrobacterium tumefaciens strain LBA4404 by heat shock (Holsters et al., Mol. Gen. Genet. 163, 181-187, 1978). A kanamycin-resistant colony was isolated, the plasmid was purified, and the entire construct was verified by PCR with the F and R primers and under the conditions described above for determining the relative orientation of the LOX sequence. The recombinant bacteria obtained were then used to infect foliar discs of tobacco, Nicotiana tabacum, lines 46-8 WT and 49-10 WT, according to already described protocols (Horsch et al., Science 227, 1229-1231, 1985).

The plants regenerated on a Murashige and Skoog (MS) medium containing 150 μg.ml⁻¹ of kanamycin were placed in a culture chamber and then in a greenhouse so as to obtain T1 seeds, by self-pollination. The transgenic lines regenerated from the parental lines 46-8 WT and 49-10 WT are called S46-x and S49-x plants, respectively.

### Example 4

### Characterization of the Primary Transformants

The presence of the 35S-LOX1 expression cassette and also the number of copies of the transgene in the genome of the regenerated plants were determined by PCR and DNA/DNA (Southern) hybridization experiments. The genomic DNA of wild-type plants or of regenerated plants which were resistant to kanamycin was prepared according to the method described by Dellaporta et al. (Dellaporta et al., Plant Mol. Biol. Rep. 1, 19-21, 1983). The entire T-DNA transferred was verified by PCR amplification using the F “sense 35S” and R “reverse LOX1” primers described above. The reaction conditions were those described above, but the amount of matrix DNA was, in this case, 800 ng of genomic DNA. The number of copies of T-DNA inserted was estimated by Southern blotting (50). The genomic DNA (15 μg) was digested with BamH1 and the digestion products were separated on agarose gel and then transferred onto nylon membrane. A CaMV 35S probe (nucleotides 6909 to 7440 of the Genbank sequence J02048) was used after labeling with [α-³²P] dCTP. The number of bands hybridizing this probe, after revelation by autoradiography, is a good indication of the number of sites and insertion of the construct.

### Example 5

### Transformation of Tobacco with the Sequence Encoding Tobacco LOX1 Under the Control of the CaMV 35S Constitutive Promoter

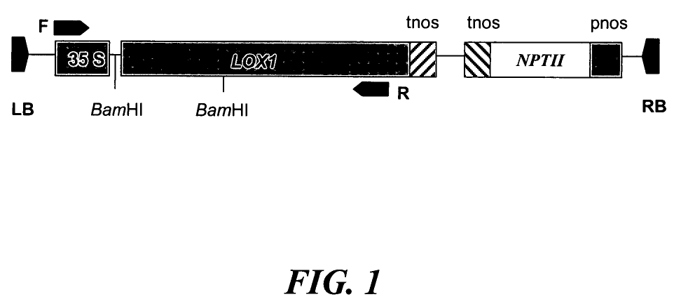

In order to constitutively express tobacco LOX1 in transgenic tobacco plants, the corresponding coding sequence was introduced in the sense orientation into the transfer DNA of the binary vector pIPM0, downstream of the CaMV 35S constitutive promoter (p35S) (FIG. 1A). This construct, called p35S-LOX1, also contains a kanamycin resistance gene (NPTII) for selecting the transformed plant cells. The 46-8 WT and 49-10 WT tobacco lines were transformed with Agrobacterium tumefaciens LBA 4404 into which the p35S-LOX1 construct has been introduced. 15 independent primary transformants S46-x derived from the 46-8 WT line were regenerated from calluses selected on a medium containing kanamycin. The letter x denotes the number of the plant obtained. Similarly, 25 independent primary transformants S49-x were regenerated from the 49-10 WT line. These plants were acclimatized in a culture chamber and then transferred into a greenhouse until flowering. The seeds corresponding to the T1 plants were obtained by self-pollination of the primary transformants.

The entire construct introduced into the genome of the transgenic plants was verified by PCR amplification using a preparation of genomic DNA of the primary transformants cultured on kanamycin, and a pair of primers, one specific for the 5′ region of the CaMV 35S promoter (F) and the other for the 3′ region of the LOX1 coding sequence (R). The amplification products were separated on agarose gel and revealed with ethidium bromide. For 10 of the 11 primary transformants analyzed, the profile obtained corresponds to a single band for which the size (2.8 kb) corresponds to the estimated size of the product. In addition, this profile is identical to that obtained with the binary vector p35S-LOX1, which suggests that at least one copy has been integrated into the genome of these transformants. On the other hand, one primary transformant does not have such a profile, although it is resistant to kanamycin, indicating an incomplete integration of the construct. The parental lines 46-8 WT and 49-10 WT, analyzed as negative controls, show no signal corresponding to the construct.

The number of copies inserted into each of the regenerated lines was estimated by Southern-type hybridization using genomic DNA digested with BamHI and a probe homologous to the CaMV 35S promoter. The transfer DNA has two BamHI sites: the first is located between the CaMV 35S promoter and the LOX1 sequence and a second is located in the LOX1 sequence. The BamHI fragments which hybridize with the radiolabeled CaMV 35S probe therefore result from a first cleavage between the CaMV 35S promoter and LOX1 and a second cleavage in the plant genome, upstream of the left border of the transfer DNA. Since the insertion of the transfer DNA into the plant genome is random, the BamHI fragments which hybridize with the radiolabeled CaMV 35S probe, obtained in the case of multiple insertions, will have sizes which depend on the position of the BamHI site in the genomic DNA and which are therefore probably different. For this reason, the number of these fragments makes it possible to evaluate the number of sites for insertion into the genome. The profiles obtained indicate that the primary transformants S46-3, S46-4, S46-26, S49-8 and S49-13 contain one copy of the transgene, whereas two copies have been inserted into the genome of the lines S49-18 and S49-28, and three copies into the genome of the lines S46-21, S49-14 and S49-30. The radiolabeled CaMV 35S probe did not hybridize with the genomic DNA corresponding to the S49-24 lines, or with that of the parental lines 46-8 WT and 49-10 WT.

### Example 6

### RNA Extraction and Analysis

The total RNA was isolated from frozen samples of T1 generation or wild-type plants. The plant material was ground in liquid nitrogen and the RNA was extracted using the Extract-all kit (Eurobio). The nucleic acid concentration was estimated by spectrophotometry. Northern blotting experiments were carried out as previously described (Rickauer et al., Planta 202, 155-162, 1997). The filters were hybridized with the radiolabeled TL-J2 probe.

### Example 7

### Analysis of LOX Transcript Accumulation in the T1 Transgenic Lines

The level of LOX expression was evaluated in the various T1 transgenic lines by measuring the LOX transcript accumulation by Northern blotting. The total RNA samples were prepared from 4-week-old young transgenic tobacco plants selected in vitro on a medium containing kanamycin. Evaluation of the respective levels of expression of the transgene in the lines was carried out by comparing the obtained profiles with the level of transcripts detected in wild-type plants, and also in a control tobacco cell suspension (negative control) or in a suspension of tobacco cells treated with Ppn elicitors (positive control). The results obtained indicate that the level of transcripts is low, or even undetectable, in the transgenic lines S46-3, S46-4, S49-8, S49-13, S49-24, S49-30 . On the other hand, the lines S46-21, S46-26, S49-14 and S49-18 exhibit a considerable accumulation of LOX transcripts, reaching, after quantification, from 30 to 66% of the level detected in the elicited tobacco cells. No LOX transcript accumulation is detected in the wild-type line or in the control tobacco cells. Introduction of the 35S promoter-LOX1 construct into the tobacco plant is therefore accompanied by considerable constitutive expression in the transgenic lines S46-21, S46-26, S49-14 and S49-18.

### Example 8

### Immunodetection of LOX

Production of an anti-tobacco LOX1 rabbit polyclonal serum: Rabbits were immunized with a fusion protein expressed in Escherichia coli and comprising the 244 C-terminal residues of tobacco LOX1 fused with Schistosoma japonicum glutathione S-transferase (GST). An XhoI fragment of pTL-J2, corresponding to nucleotides 1921 to 2888, was inserted into the XhoI site of the vector pGEX-5X-3 (Pharmacia, sequence available in Genbank under the accession number U13858) which made it possible to obtain a translational fusion with the GST coding sequence. A colony of bacteria containing the recombinant plasmid was selected and placed in culture. These bacteria were treated with 4 mM isopropylthio-β-galactoside for 16 hours at 37° C. in order to induce fusion protein production. The bacteria were harvested by centrifugation at 6000×g for 10 min and the proteins were then extracted by resuspension of the bacterial pellet in a buffered solution adjusted to 140 mM NaCl, 2.7 mM KCl, 10 mM Na₂HPO₄, 1.8 mM KH₂PO₄, pH 7.3, in a proportion of 40 μl of solution per ml of culture, and then sonication of the mixture in 3 cycles each of 1 min, on ice. The sonicate was centrifuged at 10 000×g for 5 min and the insoluble proteins contained in the centrifugation pellet were recovered and extracted in 1X SDS-PAGE loading buffer (50) at 100° C. for 10 min. After further centrifugation at 10 000×g for 5 min, the protein extract was loaded onto an 8% polyacrylamide denaturing gel. After electrophoresis and restaining with Coomassie blue, the gel was destained and the band corresponding to the fusion protein (55 kDa) was excised from the gel and used to immunize the animals (Eurogentec). One of the sera, which exhibited the best titer relative to the fusion protein and to tobacco LOX1 was selected as anti-LOX1 serum. Western blotting analysis: Dialyzed and concentrated enzyme extracts, prepared as described above, were separated by SDS-PAGE on a 10% gel at a rate of 100 μg of protein per lane and, after electrophoresis, the separated proteins were transferred onto nitrocellulose membrane by electroblotting. The Western blotting analyses were carried out according to standard protocols. The anti-LOX1 serum, at a 1:1000 dilution, was used as primary antibody, and anti-rabbit IgG goat IgGs, coupled to alkaline phosphatase (Sigmna), were used as secondary antibodies. The alkaline phosphatase enzyme activity was detected by the NBT-BCIP method.

### Example 9

### Detection of the LOX1 Protein in the T1 Transgenic Lines

Western blotting analysis was used to search for the LOX1 protein in the transgenic lines constitutively expressing the LOX1 transgene. Soluble protein extracts prepared from the aerial parts of 8-week-old plants were separated by SDS-PAGE. The LOX1 protein was detected using a rabbit polyclonal serum directed against the C-terminal portion of the LOX1 protein. Immunochemical revelation shows the presence of a single band in the lanes corresponding to the transgenic lines S46-26 and S49-18. The size of the corresponding product, between 79 and 101 kDa, is coherent with the size calculated from the primary sequence of the LOX1 protein (92 kDa). On the other hand, the LOX1 protein is not detected in the extracts prepared from the parental lines 46-8 WT and 49-10 WT. Constitutive expression of the LOX1 transgene is therefore accompanied by accumulation of the corresponding protein in the transgenic lines S46-26 and S49-18.

### Example 10

### Measurement of the LOX Activity

The samples of wild-type or transgenic plants were frozen and then ground in liquid nitrogen and homogenized in 0.25 M sodium phosphate buffer, pH 6.5, containing 5% polyvinylpolypyrrolidone, at a rate of 1 ml of buffer per g of fresh material. After thawing, the extracts were mixed by vortexing and centrifuged for 5 min at 12 000×g. The centrifugation supernatant constitutes the crude enzyme extract. Two methods for measuring the LOX activity were used.

Chromatographic method (TLC): A protocol was adapted from a method described by Caldelari and Farmer (Caldelari, D. & Farmer, E. E., Phytochemistry 47, 599-604, 1998). The LOX assay was carried out with an aliquot of the crude enzyme extract corresponding to 50 μg of proteins, in a total volume of 0.4 ml of 0.25 M sodium phosphate buffer, pH 6.5, saturated with air and containing linoleic acid labeled with ¹⁴C on the carbon in the 1-position, at a final concentration of 1.2 μM, for 30 min at 30° C. The reaction mixture was then extracted twice with a methanol/chloroform (2:1) mixture and the organic phases were concentrated under a stream of nitrogen. The extracts were separated by thin layer chromatography on silica plates, in an ether/hexane/formic acid (70:30:1) mixture. The radiolabel products derived from metabolization of the linoleic acid and also the remaining substrate were revealed by phosphorimaging. The amount of substrate remaining in each reaction was estimated by comparison with a control reaction without enzyme extract (ImageQua® software)

Spectrophotometric method: The crude enzyme extract was dialyzed and concentrated by centrifugation on an Ultrafree-4 unit (Milhpore) equipped with a Biomax 10 kDa NMWL membrane, for 30 min at 3500×g and at 4° C., and was then subjected to three washing steps by addition of 0.5 ml of 0.25 M sodium phosphate buffer, pH 6.5 and centrifugation in the same unit. The LOX activity was determined in an assay consisting of a total volume of 475 μl, by measuring the formation of conjugated dienes at λ₂₃₄ nm (ε=27 000 M⁻¹.cm⁻¹) for 4 min at 30° C., in air-saturated 0.25 M sodium phosphate buffer, pH 6.5. The linoleic acid was used as substrate at a final concentration of 820 μM. The results are expressed in nanokatal.mg⁻¹ proteins. The protein content of the aliquots tested was determined by the Bradford method (Bradford, Anal. Biochem. 72, 248-254, 1976).

### Example 11

### in vitro Conversion of Linoleic Acid by the T1 Transgenic Plants

The level of LOX activity of the transgenic plants was compared with that of the parental lines 46-8 WT and 49-10 WT by measuring, in vitro, the ability of various enzyme extracts to convert a natural substrate of this enzyme, linoleic acid. These extracts, prepared from the aerial parts of 8-week-old plants, were incubated in vitro with ¹⁴C-labeled linoleic acid. The radiolabeled products, extracted and then separated by thin layer chromatography (TLC), were revealed using a phosphorimager. From the digitized TLC image, the linoleic acid not metabolized at the end of the reaction was quantified for each lane and expressed as percentage of the linoleic acid measured in a control reaction not containing any enzyme extract. These percentages correspond to the mean of three independent repetitions. In the assays, the linoleic acid disappears almost completely in the lanes corresponding to the transgenic plants S46-26 and S49-18 with only 5 and 10% of substrate remaining at the end of the reaction, whereas, in the case of the parental lines 46-8 WT and 49-10 WT, approximately 50% of the substrate is not metabolized. In order to verify that this difference between the WT and transgenic lines is indeed due to the constitutive expression of the transgene introduced, the same reaction was carried out by preincubating the enzyme extracts with ETYA (5,8,11,14-eicosatetraynoic acid), a LOX-specific inhibitor. In this case, approximately 50% of the linoleic acid is detected at the end of the reaction both for the parental lines and for the transgenic lines. This suggests that all the lines have a LOX-independent activity capable of metabolizing part of the fatty acid introduced. When the enzyme extracts are boiled before being incubated with the substrate, between 80 and 90% of the fatty acid is extracted at the end of the reaction, showing that an enzyme reaction is indeed involved and suggesting either that part of the substrate (between 10 and 20%) is chemically degraded, or that it is not extractable under the conditions used. This experiment therefore shows that the transgenic lines S46-26 and S49-18 exhibit ETYA-sensitive linoleic acid-converting activity, which is not the case of the parental lines 46-8 WT and 49-10 WT. This shows that constitutive expression of the LOX1 transgene, and also the presence of the LOX1 protein in the transgenic lines, also results in an increase in LOX activity in these plants. This increase in activity was also measured in the S46-21 line.

### Example 12

### Inoculation of Tobacco Plants with Ppn

A method of stem inoculation of tobacco with Ppn was used. 12-week-old wild-type (46-8 WT and 49-10 WT lines) or transgenic tobacco plants were inoculated by application of a disc of mycelium onto the stem after sectioning of the apical part thereof (approximately a third of the way from the top) with a razor blade. The discs of mycelium originated from 7-day-old cultures in agar medium. Control plants were treated identically, with the exception of the application of the mycelium disc, replaced with a disc of sterile medium. The control and inoculated stems were covered with an aluminum film so as to preserve the plant tissues and the mycelium against desiccation.

### Example 13

### Observation and Measurement of Symptoms

The symptoms were observed and quantified 48 hours or 72 hours after inoculation. The stems were sectioned longitudinally and the length of the lesions was measured for each half-stem at five equidistant points, distributed over the entire width of the section. The lesion length used for each individual corresponds to the mean of these 10 measurements.

### Example 14

### Measurement of LOX1 Transcript Accumulation and of LOX Specific Activity in the Stems of the T1 Transgenic Lines

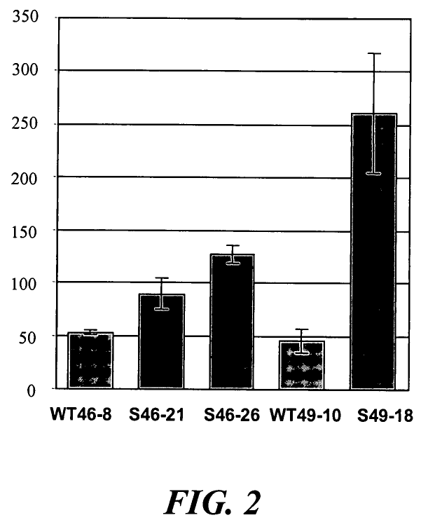

The method selected for testing the interaction between tobacco and the pathogenic microorganism Ppn consists in inoculating the stem with Ppn mycelium, after sectioning the apex of the plant. As a prelude to this experiment, the level of expression of the transgene and also the LOX specific activity in the stems of the transgenic lines S46-21, S46-26 and S49-18 were compared with those observed in the parental lines 46-8 WT and 49-10 WT. For each line, total RNAs were prepared from a pool of 3 pieces of stem each originating from an independent plant. The result of hybridization with a radiolabeled LOX1 probe confirms LOX transcript accumulation in the stems of the transgenic lines S46-21, S46-26 and S49-18, whereas no LOX transcript is detected in the parental lines 46-8 WT and 49-10 WT. The LOX specific activity was also measured in this organ, using concentrated and dialyzed enzyme extracts. The analysis was carried out in a spectrophotometer by measuring the appearance of fatty acid hydroperoxides at 234 nm. For each line studied, 3 independent measurements were taken. The results obtained, pooled in a histogram (FIG. 2), indicate that the LOX specific activity measured in the transgenic lines S46-21, S46-26 and S49-18 is 1.8 to 5 times greater than the level of activity measured in the parental lines 46-8 WT and 49-10 WT. In addition, these levels of activity reach 25% (S46-21) and 70% (S49-18) of the level of LOX activity measured in tobacco cells elicited for 24 hours (353.8 nkat.mg⁻¹ protein, data not shown). This analysis therefore confirms that the constitutive expression of the LOX transgene, and also the increase in LOX activity, measured in the transgenic plants also characterizes the stems.

### Example 15

### Analysis of the Interaction Between Ppn and the T1 Transgenic Lines Having Constitutive LOX Activity

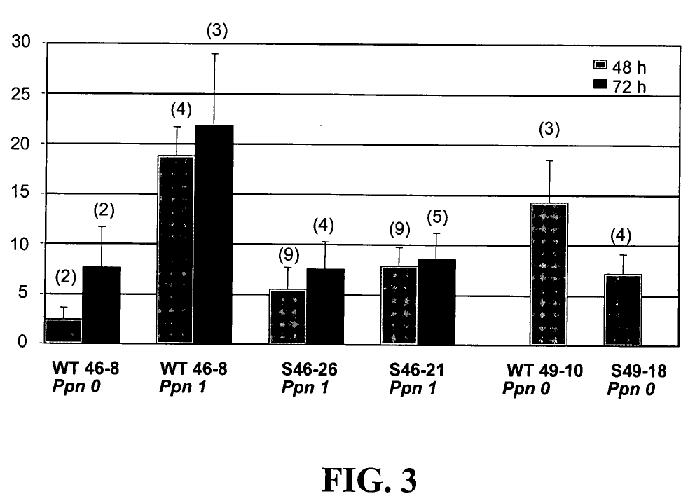

In order to examine the consequences of constitutive LOX1 expression in the transgenic lines S46-21, S46-26 and S49-18 on their interaction with Ppn, 12-week-old plants of these lines were inoculated with this pathogenic agent at the level of the stems. The symptoms obtained after inoculation with a virulent race of Ppn were compared with those observed during a compatible interaction involving the corresponding parental line. Thus, the lines S46-21, S46-26 and 46-8 WT were inoculated with race 1 of Ppn, whereas the lines S49-18 and 49-10 WT were inoculated with race 0 of Ppn. An incompatibility control was carried out by inoculating the line 46-8 WT with race 0 of Ppn. The symptoms obtained 48 hours or 72 hours after inoculation were observed on longitudinal sections of the stems and the lesions were measured (FIG. 3).

The symptoms observed on the parental lines 46-8 WT and 49-10 WT are typical of tobacco/Ppn interactions; the line 46-8 WT, inoculated with race 0 of Ppn, exhibits dry and localized lesions characteristic of an incompatible interaction. On the other hand, the long brown macerated lesions observed in the 46-8 WT/Ppn 1 and 49-10 WT/Ppn 0 interactions reflect the colonization of the stem by the pathogenic agent and are typical of compatible interactions. In comparison with the latter, the lesions measured in the transgenic lines, inoculated with the same virulent race as that used with the corresponding parental line, are clearly reduced. In the two transgenic lines selected, S46-21 and S46-26, inoculation with race 1 of the fungus does not cause the formation of these long macerated lesions. Lesions are observed which are much more reduced than in the compatible case, but also much less macerated. This difference is also observed when the compatible interaction 49-10 WT/Ppn 0 (Ppn 0-sensitive wild-type line) and the interaction between the transgenic line S49-18, which is derived from the line 49-10 WT, and Ppn 0 are compared. It is observed that the lesions caused during the S46-26/Ppn 1 interaction resemble more closely the necroses which appear during an incompatible interaction (46-8 WT/Ppn 0) than the lesions which accompany the colonization of the plant tissues by the fungus in the case of a compatible interaction (46-8 WT/Ppn 1). For example, the lesions obtained 48 hours after inoculation in the S46-21/Ppn 1 and S46-26/Ppn 1 interactions are respectively 3.4 and 2.4 times shorter than those measured in the compatible interaction 46-8 WT/Ppn 1. For the S49-18 line, inoculation with race 0 of Ppn causes lesions twice as short as those observed for the parental line 49-10 WT inoculated with the same race of the fungus. All these results show that constitutive LOX expression in the transgenic lines is accompanied by a clear limitation of the progression of the fungus.

Besides the reduction in size of the lesions observed in the transgenic lines inoculated with a virulent race of Ppn, the nature thereof is also modified. The lesions obtained in the S46-26/Ppn 1 interaction are not macerated as in the compatible interaction 46-8 WT/Ppn 1, but are rather dry as in the incompatible interaction 46-8 WT/Ppn 0. This shows that the constitutive LOX activity measured in the transgenic lines S46-21, S46-26 and S49-18 actively contribute to the resistance of tobacco to Ppn.

### Example 16

### Analysis of the Interaction Between Ppn and the T1 Transoenic Plants Having Constitutive LOX Activity—Root Inoculation

Plants constitutively expressing tobacco LOX1 were previously obtained. In a stem inoculation test, the plants showed reduced sensitivity to Phytophthora parasitica var. nicotianae (Ppn) in comparison with the wild-type line from which they are derived. The behavior of these plants was studied in a root inoculation test.

Wild-type tobacco plants (Nicotiana tabacum L.) of the 46-8 line (46-8 WT), characterized by the presence of a locus for resistance to race 0 of Ppn, and plants of the sense-lipoxygenase S46-21 transgenic line, derived from the 46-8 WT line, were sown in vitro. The seeds of wild-type or transgenic lines were sterilized and sown in Petri dishes, on solid MS medium at a rate of approximately 30 seeds per dish, by intercollating a disc of synthetic cloth between the medium and the seeds. The dishes were placed in a sloping position so as to orient the growth of the roots. After 3 weeks of growth in vitro (40% hygrometry, constant temperature 25° C., light 60 μmol.m⁻².s⁻¹: 16h, dark: 8h), the discs of cloth supporting the plants were transferred into new Petri dishes containing liquid MS medium and glass beads, and the plants were returned to the culture chamber for 2 days, before inoculation. The Ppn strains used correspond to isolates 1156 (race 0) and 1452 (race 1). The Ppn mycelium was grown in the dark on a solid synthetic medium.

A method for root inoculation of tobacco using a suspension of Ppn zoospores was used. A Ppn mycelium colony obtained on V8 medium was placed under conditions of deficiency on agar-water for 4 days, and the zoospores were then released by cold shock (30 min at 15° C. and then 30 min at ambient temperature) in 10 ml of water. After counting, the zoospore suspension is adjusted to 4000 spores/ml. For each dish, the liquid MS medium is removed and replaced with the spore suspension. The plants exhibiting no symptoms of disease are counted after 6 to 11 days, and the percentage survival (plants free of symptoms/total plants) is calculated for each combination.

In order to examine the consequences of constitutive LOX1 expression in tobacco on the interaction with Ppn, 3-week-old plants of the S46-21 transgenic line were inoculated at the level of the roots with virulent race 1 of this pathogenic antigen. The outcome of the inoculated plants was compared with that of wild-type plants of the corresponding parental line 46-8 WT, inoculated with this same race (compatible interaction). An incompatibility control was carried out, by inoculating the 46-8 WT line with race 0 of Ppn under the same conditions, as was a noninoculated control. The plants were observed 6 or 11 days after inoculation in a first experiment, and 7 days after inoculation in a second experiment. The plants free of symptoms of disease were counted and the percentage survivals were calculated (Table 1).

The 46-8 WT line inoculated with race 0 of Ppn exhibits no symptoms and the percentage survival is very close to 100%. On the other hand, in the 46-8 WT/Ppn 1 interaction, the colonization of the plants by the pathogenic antigen results in considerable mortality and a low percentage survival (20 to 24%). In comparison with this compatible interaction, the plants of the sense LOX S46-21 transgenic line, inoculated with the same virulent race as that used with the 46-8 WT line of origin, exhibit a much higher percentage survival, in two independent experiments (survival rate 80 to 88%). All these results confirm that constitutive LOX expression tobacco is accompanied by a notable reduction in sensitivity to Ppn.

TABLE 1
Percentage survival of the wild-type or sense-LOX transgenic plants,
after inoculation with virulent race 1 of Ppn, and comparison with the
incompatible interaction 46-8 WT/race 0

| | Total | | |
|---|---|---|---|
| | number of | Survival rate^{a} | |
| | plants | D6 | D11 |
| EXPERIMENT N° 1 | | | |
| 46-8 WT/Ppn 0 (incompatible) | n = 58 | 100% | 100% |
| 46-8 WT/Ppn 1 (compatible) | n = 55 | 24.0% | 20.0% |
| S46-21/Ppn 1 | n = 54 | 88.8% | 88.8% |

D7

| | | |
|---|---|---|
| EXPERIMENT N° 2 | | |
| 46-8 WT control | n = 47 | 95.7% |
| 46-8 WT/Ppn 1 (compatible) | n = 82 | 21.9% |
| S46-21/Ppn 1 | n = 65 | 80.0% |

^{a}Survival rate = % number of plants without symptoms of disease/total number of plants.

### Cited Documents

All sequences, patents, patent applications or other publications cited anywhere in this specification are herein incorporated in their entirety by reference to the same extent as if each individual sequence, publication, patent, or patent application was specifically and individually indicated to be incorporated by reference.

1. Brash, A. R., Ingram, C. D. & Harris, T. M. (1987) Analysis of a specific oxygenation reaction of soybean lipoxygenase-1 with fatty acids esterified in phospholipids, Biochemistry 26, 5465-5471.

2. Maccarrone, M., van Aarle, P. G., Veldink, G. A. & Vliegenthart, J. F. (1994) In vitro oxygenation of soybean biomembranes by lipoxygenase-2, Biochim. Biophys. Acta 1190, 164-169.

3. Feussner, I., Bachmann, A., Hohne, M. & Kindl, H. (1998) All three acyl moieties of trilinolein are efficiently oxygenated by recombinant His-tagged lipid body lipoxygenase in vitro, FEBS Lett. 431, 433-436.

4. Blée, E. (1998) Phytooxylipins and plant defense reactions, Prog. Lipid Res. 37, 33-72.

5. Rosahl, S. (1996) Lipoxygenases in plants—their role in development and stress response, Z. Naturforsch., C: Biosci. 51, 123-138.

6. Homung, E., Walther, M., Kuhn, H. & Feussner, I. (1999) Conversion of cucumber linoleate 13-lipoxygenase to a 9-lipoxygenating species by site-directed mutagenesis, Proc. Natl. Acad. Sci. U.S.A. 96, 4192-4197.

Kuhn, H. & Thiele, B. J. (1999) The diversity of the lipoxygenase family. Many sequence data but little information on biological significance, FEBS Lett. 449, 7-11.

8. Galliard, T. & Chan, H. W. S. (1980) in The Biochemistry of Plants A comprehensive treatise, ed. Stumpf, P. K. (Academic Press, INC, New York), Vol. 4, pp. 131-161.

9. Slusarenko, A. J. (1996) in Lipoxygenase and lipoxygenase pathway enzymes, ed. Piazza, G. J. (AOCS Press, Champain, Ill.), pp. 176-197.

10. Croft, K. P. C., Jüttner, F. & Slusarenko, A. J. (1993) Volatile products of the lipoxygenase pathway evolved from Phaseolus vulgaris (L.) leaves inoculated with Pseudomonas syringae pv. phaseolicola, Plant Physiol. 101, 13-24.

11. Bate, N. J. & Rothstein, S. J. (1998) C6-volatiles derived from the lipoxygenase pathway induce a subset of defense-related genes, Plant J. 16, 561-569.

12. Reymond, P. & Farmer, E. E. (1998) Jasmonate and salicylate as global signals for defense gene expression, Current Opinion in Plant Biology 1, 404-411.

13. McConn, M., Creelman, R. A., Bell, E., Mullet, J. E. & Browse, J. (1997) Jasmonate is essential for insect defense in Arabidopsis, Proc. Natl. Acad. Sci. U.S.A. 94, 5473-5477.

14. Staswick, P. E., Yuen, G. Y. & Lehman, C. C. (1998) Jasmonate signaling mutants of Arabidopsis are susceptible to the soil fungus Pythium irregulars, Plant J. 15, 747-754.

15. Howe, G. A., Lightner, J., Browse, J. & Ryan, C. A. (1996) An octadecanoid pathway mutant (JL5) of tomato is compromised in signaling for defense against insect attack, Plant Cell 8, 2067-2077.

16. Itoh, A. & Howe, G. A. (2001) Molecular cloning of a divinyl ether synthase: Identification as a CYP74 cytochrome P-450, J. Biol. Chem. 276, 3620-3627.

17. Weber, H., Chetelat, A., Caldelari, D. & Farmer, E. E. (1999) Divinyl ether fatty acid synthesis in late blight-diseased potato leaves, Plant Cell 11, 485-494.

18. Schweizer, P., Felix, G., Buchala, A., Muller, C. & Metraux, J. P. (1996) Perception of free cut in monomers by Plant Cells, Plant J. 10, 331-341.

19. Rickauer, M., Fournier, J., Pouénat, M. L., Berthalon, E., Bottin, A. & Esquerre-Tugaye, M. T. (1990) Early changes in ethylene synthesis and lipoxygenase activity during defense induction in tobacco cells, Plant Physiology and Biochemistry 28, 647-653.

20. Fournier, J., Pouénat, M. L., Rickauer, M., Rabinovitch-Chable, H., Rigaud, M. & Esquerré-Tugayé, M. T. (1993) Purification and characterisation of elicitor-induced lipoxygenase in tobacco cells, Plant J. 3, 63-70.

21. Véronési, C., Fourniér, J., Rickauer, M., Marolda, M. & Esquerré-Tugayé, M. T. (1995) Nucleotide sequence of an elicitor-induced tobacco lipoxygenase cDNA (Genbank X84040), Plant Physiol. 108, 1342.

22. Véronési, C., Rickauer, M., Fournier, J., Pouénat, M. L. & Esquerré-Tugayé, M. T. (1996) Lipoxygenase gene expression in the tobacco-Phytophthora parasitica nicotianae interaction, Plant Physiol . 112, 997-1004.

23. Koch, E., Meier, B. M., Eiben, H. G. & Slusarenko, A. J. (1992) A lipoxygenase from leaves of tomato (Lycopersicum esculentum Mill) is induced in response to plant pathogen Pseudomonas, Plant Physiol. 99, 571-576.

24. Peng, Y. L., Shirano, Y., Ohta, H., Hibino, T., Tanaka, K. & Shibata, D. (1994) A novel lipoxygenase from rice. Primary structure and specific expression upon incompatible infection with rice blast fungus, J. Biol. Chem. 269, 3755-3761.

25. Kolomiets, M. V., Chen, H., Gladon, R. J., Braun, E. J. & Hannapel, D. J. (2000) A leaf lipoxygenase of potato induced specifically by pathogen infection, Plant Physiol. 124, 1121-1130.

26. Véronési, C. (1995) (Université Paul Sabatier—Toulouse III, Toulouse), pp.90.

27. Villalba Mateos, F., Rickauer, M. & Esquerré-Tugayé, M. T. (1997) Cloning and characterization of a cDNA encoding an elicitor of Phytophthora parasitica var. nicotianae that shows cellulose-binding and lectin-like activities, Mol. Plant-Microbe Interact. 10, 1045-1053.

28. Mercier, L., Lafitte, C., Borderies, G., Briand, X., Esquerre-Tugaye, M. T. & Fournier, J. (2001) The algal polysaccharide carrageenans can act as an elicitor of plant defence, The New Phytologist 149, 43-51.

29. Rickauer, M., Brodschelm, W., Bottin, A., Véronési, C., Grimal, H. & Esquerré-Tugayé, M. T. (1997) The jasmonate pathway is involved differentially in the regulation of different defence responses in tobacco cells, Planta 202, 155-162.

30. Rancé, I. (1997) (UniversitéParis XI—Orsay), pp.111.

31. Rancé, I., Fournier, J. & Esquerré-Tugayé, M. T. (1998) The incompatible interaction between Phytophthora parasitica var. nicotianae race 0 and tobacco is suppressed in transgenic plants expressing antisense lipoxygenase sequen\overline{c}es, Proc. Natl. Acad. Sci. U.S.A. 95, 6554-6559.

32. Rustérucci, C., Montillet, J. L., Agnel, J. P., Battesti, C., Alonso, B., Knoll, A., Bessoule, J. J., Etienne, P., Suty, L., Blein, J. P. & Triantaphylides, C. (1999) Involvement of lipoxygenase-dependent production of fatty acid hydroperoxides in the development of the hypersensitive cell death induced by cryptogein on tobacco leaves, J. Biol. Chem. 274, 36446-36455.

33. Göbel, C., Feussner, I., Schmidt, A., Scheel, D., Sanchez-Serrano, J., Hamberg, M. & Rosahl, S. (2001) Oxylipin profiling reveals the preferential stimulation of the 9-lipoxygenase pathway in elicitor-treated potato cells, J. Biol. Chem. 276, 6267-6273.

34. Royo, J., Leon, J., Vancanneyt, G., Albar, J. P., Rosahl, S., Ortego, F., Castanera, P. & Sanchez-Serrano, J. J. (1999) Antisense-mediated depletion of a potato lipoxygenase reduces wound induction of proteinase inhibitors and increases weight gain of insect pests, Proc. Natl. Acad. Sci. U S. A. 96, 1146-1151.

35. Deng, W., Graybum, W. S., Hamilton-Kemp, T. R., Collins, G. B. & Hildebrand, D. F. (1992) Expression of soybean-embryo lipoxygenase 2 in transgenic tobacco tissue, Planta 187, 203-208.

36. Hause, B., Weichert, H., Hohne, M., Kindl, H. & Feussner, I. (2000) Expression of cucumber lipid-body lipoxygenase in transgenic tobacco: lipid-body lipoxygenase is correctly targeted to seed lipid bodies, Planta 210, 708-714.

37. Bell, E., Creelman, R. A. & Mullet, J. E. (1995) A chloroplast lipoxygenase is required for wound-induced jasmonic acid accumulation in Arabidopsis, Proc. Natl. Acad. Sci. U.S.A. 92, 8675-8679.

38. Maccarrone, M., Hilbers, M. P., Veldink, G. A., Vliegenthart, J. F. & Finazzi Agro, A. (1995) Inhibition of lipoxygenase in lentil protoplasts by expression of antisense RNA, Biochim. Biophys. Acta 1259, 1-3.

39. Grechkin, A. (1998) Recent developments in biochemistry of the plant lipoxygenase pathway, Prog. Lipid Res. 37, 317-352.

40. Laudert, D., Pfannschmidt, U., Lottspeich, F., Hollander-Czytko, H. & Weiler, E. W. (1996) Cloning, molecular and functional characterization of Arabidopsis thaliana allene oxide synthase (CYP 74), the first enzyme of the octadecanoid pathway to jasmonates, Plant Mol. Biol. 31, 323-335.

41. Bate, N. J., Sivasankar, S., Moxon, C., Riley, J. M., Thompson, J. E. & Rothstein, S. J. (1998) Molecular characterization of an Arabidopsis gene encoding hydroperoxide lyase, a cytochrome P-450 that is wound inducible, Plant Physiol. 117, 1393-1400.

42. Howe, G. A., Lee, G. I., Itoh, A., Li, L. & DeRocher, A. E. (2000) Cytochrome P450-dependent metabolism of oxylipins in tomato. Cloning and expression of allene oxide synthase and fatty acid hydroperoxide lyase, Plant Physiol. 123, 711-24.

43. Helgeson, J. P., Kemp, J. D., Haberlach, G. T. & Maxwell, D. P. (1972) A tissue culture system for studying disease resistance : the black shank disease in tobacco callus culture, Phytopath. 62, 1439-1443.

44. Rancé, I., Tian, W. Z., Mathews, H., De Kochko, A., Beachy, R. N. & Fauquet, C. M. (1994) Partial dessication of mature embryo-derived calli, a simple treatment that dramatically enhances the regeneration ability of indica rice., Plant Cell Report 13, 647-651.

45. Hendrix, J. W. & Apple, J. L. (1967) Stem resistance to Phytophthora parasitica var. nicotianae in tobacco derived from Nicotiana longiflora and Nicotiana plumbaginifolia, Tobacco Science 11, 148-150.

46. Keen, N. T. (1975) Specific elicitors of plant phytoalexin production: determinants of race specificity in pathogens?, Science 187, 74-75.

47. Holsters, M., de Waele, D., Depicker, A., Messens, E., van Montagu, M. & Schell, J. (1978) Transfection and transformation of Agrobacterium tumefaciens, Mol. Gen. Genet. 163, 181-187.

48. Horsch, R. B., Fry, J. E., Hoffmann, N. L., Eicholtz, D., Rogers, S. G. & Fraley, R. T. (1985) A simple and general method for transferring genes into plants, Science 227, 1229-1231.

49. Dellaporta, S. L., Wood, J. & Hicks, J. B. (1983) A plant DNA minipreparation: version II., Plant Mol. Biol. Rep. 1, 19-21.

50. Sambrook, J., Fritsh, E. F. & Maniatis, T. (1989) Molecular cloning: a laboratory manual Cold Spring Harbor Laboratory Press, New-York).

51. Caldelari, D. & Farmer, E. E. (1998) A rapid assay for the coupled cell free generation of oxylipins, Phytochemistry 47, 599-604.

52. Bradford, M. M. (1976) A rapid and sensitive method for the quantitation of microgram quantities of protein utilizing the principle of protein-dye binding, Anal. Biochem. 72, 248-254.

53. Seo, H. S., Song, J. T., Cheong, J.-J., Lee, Y.-H., Lee, Y.-W., Hwang, I., Lee, J. S. & Choi, Y. D. (2001) Jasmonic acid carboxyl methyltransferase: A key enzyme for jasmonate-regulated plant responses, Proc. Natl. Acad. Sci. U.S.A. 98, 4788-4793.

We claim:

1. A method for reducing plant sensitivity to at least one pathogen or at least one herbivore comprising overexpressing a lipoxygenase gene in said plant.

2. The method of claim 1, wherein said expression of said lipoxygenase gene in said plants is constitutive.

3. The method of claim 1, wherein the expressed lipoxygenase has 9-lipoxygenase activity.

4. The method of claim 1, wherein said lipoxygenase gene is a plant lipoxygenase gene.

5. The method of claim 1, wherein said lipoxygenase gene is a Solanacea plant lipoxygenase gene.

6. The method of claim 1, wherein said lipoxygenase gene comprises a nucleic acid sequence that encodes a protein that is at least 80% homologous to the lipoxygenase of SEQ ID NO:1 and has lipoxygenase activity.

7. The method of claim 6, wherein said lipoxygenase gene comprises a nucleic acid sequence that encodes the amino acid sequence of SEQ ID NO:1.

8. The method of claim 1 further comprising contacting the genome of said plant with an expression cassette comprising a sequence encoding said lipoxygenase gene operably linked to an expression control sequence that is functional in plants under conditions that permit integration of said expression cassette into the genome of said plant and wherein said expression cassette is integrated into the genome of said plant.

9. The method of claim 8, wherein said expression control sequence is an expression control sequence which is constitutive in plants.

10. The method of claim 9, wherein said constitutive expression control sequence is the cauliflower mosaic virus 35S promoter.

11. The method of claim 1, wherein said lipoxygenase genes is overexpressed in the stems, the leaves, and the roots of said plant.

12. An expression cassette which is functional in plant cells and plants comprising an expression control sequence having constitutive activity in plants operably linked to a nucleic acid encoding a protein that is at least 90% homologous to SEQ ID NO:1 wherein said protein has lipoxygenase activity.

13. The expression cassette of claim 12, wherein said nucleic acid encodes a protein having 9-lipoxygenase activity.

14. The expression cassette of claim 12, wherein said nucleic acid encodes the lipoxygenase of SEQ ID NO:1.

15. The expression cassette of claim 12, wherein said expression control sequence is the cauliflower mosaic virus 35S promoter.

16. A vector comprising an expression cassette which is functional in plant cells and plants comprising an expression control sequence having constitutive activity in plants operably linked to a nucleic acid encoding a protein that is at least 90% homologous to SEQ ID NO:1 wherein said protein has lipoxygenase activity.

17. A transformed plant cell comprising an expression cassette which is functional in plant cells and plants comprising an expression control sequence having constitutive activity in plants operably linked to a nucleic acid encoding a protein that is at least 90% homologous to SEQ ID NO:1 wherein said protein has lipoxygenase activity.

18. A transformed plant cell comprising a vector comprising an expression cassette which is functional in plant cells and plants comprising an expression control sequence having constitutive activity in plants operably linked to a nucleic acid encoding a protein that is at least 90% homologous to SEQ ID NO:1 wherein said protein has lipoxygenase activity.

19. A transformed plant comprising an expression cassette which is functional in plant cells and plants comprising an expression control sequence having constitutive activity in plants operably linked to a nucleic acid encoding a protein that is at least 90% homologous to SEQ ID NO:1 wherein said protein has lipoxygenase activity.

20. A transformed plant comprising a vector comprising an expression cassette which is functional in plant cells and plants comprising an expression control sequence having constitutive activity in plants operably linked to a nucleic acid encoding a protein that is at least 90% homologous to SEQ ID NO:1 wherein said protein has lipoxygenase activity.

21. A transformed plant, comprising the transformed plant cell of claim 17.
